(19) United States
(12) Patent Application Publication
(10) Pub. No.: US 2012/0251576 A1
(43) Pub. Date: Oct. 4, 2012

(54) Treatment of Nasal and Sinus Disorders

(76) Inventor: Ira Sanders, North Bergen, NJ (US)

(21) Appl. No.: 13/516,338

(22) PCT Filed: Dec. 15, 2010
(86) PCT No.: PCT/US10/60558

§ 371 Date: Jun. 15, 2012

Related U.S. Application Data
(60) Provisional application No. 61/286,513, filed on Dec. 15, 2009.

(51) Int. Cl. A61K 39/08; A61P 27/02; A61P 11/00; A61P 25/06; A61P 11/02; A61P 11/06

(52) U.S. Cl. 424/239.1

(57) ABSTRACT

Disclosed herein are compositions and methods for treating nasal and sinus conditions, and symptoms thereof, with botulinum toxin (BoNT).

### CLAIM OF PRIORITY

This application claims priority to U.S. Provisional Application No. 61/286,513, titled “Treatment of nasal and sinus disorders” filed Dec. 15, 2009, the disclosure of which is incorporated by reference in its entirety.

### STATEMENT REGARDING REFERENCES

All references cited herein are hereby incorporated by reference in their entirety.

### BACKGROUND OF THE INVENTION

### Anatomy of the Nose

The external nose begins at the forehead and passes inferiorly to the lower lip. The upper half of the nose is narrow and composed of bone, the lower half of the nose is cartilage and projects forward and widens laterally to form the nostrils.

The nasal bones are in the midline, lateral to them are the nasal process of the maxillary bone, then the lacrimal bone, and then on the side of the eye, the ethmoid bone. These bony plates are separated by small gaps called sutures which are relatively minor barriers for drugs to pass. The bony plates also have perforations for blood vessels and nerves which also serve as soft tissue conduits for drug delivery. Thin cartilage plates give the nasal alae their shape. These cartilages have gaps between them that drugs can diffuse through if applied topically or when delivered by injection.

The nasal cavity is divided into two symmetrical halves by the nasal septum, a central partition of bone and cartilage; each side opens at the face via the nostrils and connects with the mouth at the nasopharynx. The nasal vestibule is the initial airspace, and its interior surface is covered with skin. After the nasal vestibule air passes into the nasal cavity which is largely covered with typical respiratory epithelium, except superiorly where specialized olfactory epithelium is present. The nasal mucosa is continuous with the mucosa of the sinuses at the sinus openings (ostia).

### Physiology of the Nose

The major functions of the nose are to warm, humidify and filter inspired air. In addition specialized olfactory mucosa sample the inspired air for its smell.

In the main nasal airway, the passages are narrow, normally only 1-3 mm wide, and this narrows structure enables the nose to carry out its main functions. During inspiration, the air comes into close contact with the nasal mucosa and particles such as dust and bacteria are trapped in the mucous. Additionally, the inhaled air is warmed and moistened as it passes over the mucosa; and to perform these functions there is a high blood supply in the nasal epithelium. The lateral wall of the nasal cavity includes three folded structures called turbinates: the superior, the median and the inferior. The turbinates channel airflow and can swell to decrease airflow.

The nasal respiratory epithelium is generally described as a pseudo-stratified ciliated columnar epithelium. The four main types of cells seen in the respiratory epithelium are ciliated columnar cells, non-ciliated columnar cells, goblet cells and basal cells. The proportions of the different cell types vary in different regions of the nasal cavity. In the lower turbinate area, about 15-20% of the total numbers of cells are ciliated and 60-70% is non-ciliated epithelial cells. The role of the ciliated cells is to transport mucus towards the pharynx. The numbers of ciliated cells increase towards the nasopharynx with a corresponding decrease in non-ciliated cells. Both columnar cell types have numerous (about 300-400 per cell) microvilli. The large number of microvilli increases the surface area and this is one of the main reasons for the relatively high absorptive capacity of the nasal cavity. The high number of non-ciliated cells in the front of the nasal cavity suggests that this area is relatively more important for absorption across the nasal epithelium.

Basal cells, which vary greatly in both number and shape, never reach the airway lumen. These cells are poorly differentiated and act as stem cells to replace other epithelial cells. About 5-15% of the mucosal cells in the turbinates are goblet cells, which contain numerous secretory granules filled with mucin. In conjunction with the nasal glands; the goblet cells produce secretions, which form the mucus layer.

It has been reported that particle sizes greater than 10 μm are deposited in the nasal cavity. Particles that are 2 to 10 μm can be retained in the lungs, and particles of less than 1 μm are exhaled.

Nasal disorders

Rhinitis is one of the most common disorders in medicine. Approximately 60 million Americans suffer from rhinitis each year. The symptoms of rhinitis are 1) rhinorrhea, post-nasal drip and runny nose; 2) congestion; and 3) sneezing and itching in allergic rhinitis.

The congestion of rhinitis can lead to edema and polyps. In addition it can cause obstruction of the eustachian tubes, causing serious otitis media. Moreover, it can obstruct the opening of the sinuses, leading to bacterial sinusitis.

The annual cost for rhinitis therapy is approximately $10 B in the United States alone. These therapies include nasal sprays (Rhinocort®, Flonase®, Beconase®, Astelin®, Atrovent®), antihistamines (Allegra®, Claritin®, Zyrtec®, Singulair®) as well as many other prescription and over the counter medications.

Typical therapy for rhinitis requires the use of more than one drug to have a therapeutic effect. Some of these are used several times a day. However, the benefits are often unpredictable while the drugs can cause bothersome side effects. As allergic diseases are increasing in industrialized countries their related disorders and complications are also increasing. For example, deaths from asthma are actually higher now than 20 years ago. In addition all major pharmaceutical companies have at least one major drug product in this category, and many of these have lost or about to lose their patent protection. In summary, patients, physicians, and the pharmaceutical industry are all looking for safe and effective therapies that are predictable and cause less patient discomfort.

### Botulinum Neurotoxin (BoNT)

Botulinum toxin (“BoNT”) blocks the signals passing from nerves to muscles and causes muscle paralysis. When food contaminated with the Clostridium botulinum bacteria is eaten, it causes botulism, which is a progressive paralysis leading to respiratory failure and death. In botulism the toxin enters the circulation and is distributed systemically. However, it has long been known that local injection of small amounts of BoNT can exert local effects without systemic spread.

In the late 1960s, Dr. Alan B. Scott, of the Smith-Kettlewell Eye Research Foundation in collaboration with Mr. Edward J. Schantz, PhD, Director of food microbiology and toxicology at the University of Wisconsin was the first to recognize the possible clinical usefulness of BoNT. He showed that small injections into the spastic eyelid muscles of patients could eliminate the spasms. The unique aspect of botulinum toxin injections was the unusually long duration of its effect, lasting between 3-6 months. Since then BoNT has been applied to a variety of spastic muscle disorder such as blepharospasm, facial spasm, writers cramp, spastic dysphonia, and cervical dystonia. As the amount of toxin injected is so small that it is not recognized by the body's immune system, patients can receive repeat injections indefinitely. Side effects are rare with BoNT and the head of biological drugs at the FDA has stated that it is one of the safest biological products on the market.

The use of botulinum toxin to treat rhinitis was introduced with U.S. Pat. No. 5,766,605 which taught the intranasal use of botulinum toxin to treat rhinorrhea in a condition called vasomotor rhinitis. This method was validated by preclinical and clinical studies incorporated here in their entirety by reference (Shaari et al, Otolaryngol Head Neck Surg. 1995 April; 112(4):566-71.; Rohrbach et al, ORL J Otorhinolaryngol Relat Spec. 2001 November-December; 63(6):382-4; Lin Chuang Er Bi Yan Hou Ke Za Zhi. 2003 November; 17(11):643-5). U.S. patent application Ser. No. 10/535,504 to I. Sanders discloses the treatment of allergic rhinitis and other conditions with BoNT. Subsequent research has confirmed that BoNT is very effective at treating allergic rhinitis when injected into the nasal mucosa (Otolaryngol Head Neck Surg. 2008 September; 139(3):367-71).

There is a need in medicine for improved drugs and drug delivery methods for nasal and sinus diseases. Disclosed herein are new compositions of nasal drugs and methods of delivering these drugs.

Currently, BoNT has been applied by injections into the mucosa of the nasal cavity or by packing the nose with gauze soaked in BoNT solution. Injections are undesirable due to pain, bleeding and risk of contamination. Gauze soaked in BoNT can leak out of the nose and the actual amount delivered is unpredictable.

### SUMMARY OF THE INVENTION

It is an object of this invention to provide a method of treating a nasal or sinus condition, or symptom thereof, comprising administering a therapeutically effective amount of botulinum toxin (BoNT) to a selected site outside the nasal cavity of a mammal suffering from a nasal or sinus condition, or a symptom thereof, wherein the nasal or sinus condition, or symptom thereof, is treated.

In is a further object of the invention to provide a method of treating a nasal or sinus condition, or symptom thereof, comprising administering a therapeutically effective amount of botulinum toxin (BoNT) by jet or pressure injection to a selected site of a mammal suffering from a nasal or sinus condition, or symptom thereof, wherein the nasal or sinus condition, or symptom thereof, is treated.

In certain embodiments, the BoNT is administered by needle injection, microneedle injection, jet or pressure injection, or topical application.

In certain embodiments, the selected site is selected from the group consisting of the nasal skin overlying the nasal cartilates, nasal bones, maxillary bones, lacrimal bones, ethmoid bone, or frontal bones; the nasal tip; the external nares; the nasal alae; the palpated boy margin of the pyriform aperture; the lacrimal sac or duct; under the lip; the gingival mucosa; the sphnopalatine foramen; and the infraorbital foramen.

In certain embodiments, the BoNT is delivered by needle injection through nasal, ethmoid, frontal maxillary, or palatine bones.

In other embodiments, the BoNT is delivered by jet or pressure injection across the anterior walls of the maxillary sinus.

In some embodiments, the nasal or sinus condition is selected from the group consisting of rhinitis, infectious rhinitis, allergic rhinitis, sinusitis, asthma, COPD, migraine headache, impaired cerebral blood flow, sleep breathing disorders, and ocular disorders.

In certain embodiments, the symptom is selected from the group consisting of nasal congestion, sneezing, rhinorrhea, postnasal drip, nasal or sinus pain, headache, coughing, wheezing, itching, redness, thickened nasal mucosa, and nasal polyp.

In some embodiments, the dose of BoNT is from about 0.1 to about 100,000 units. In further embodiments, the dose of BoNT is from about 1 to about 100 units.

In certain embodiments, the BoNT is combined with a skin permeability enhancing agent. In some embodiments, the BoNT is combined with a gel. In further embodiments, the viscosity of the gel increases after administration. In further embodiments, the gel is a temperature sensitive poloxamer.

In certain embodiments, the BoNT is applied with a penetration enhancing agent. In some embodiments, the penetration enhancing agent is selected from the group consisting of allergen, histamine, and electrical stimulation.

In some embodiments, a Clostridia neurotoxin (CnT) other than BoNT is used in place of BoNT. In other embodiments, another CnT is used in combination with BoNT.

### DESCRIPTION OF THE FIGURES

FIGS. 1A, 1B, 1C, and 1D depict various aspects of the nasal anatomy. FIG. 1D identifies the glabella 1, nasion 2, rhinion 3, alar sidewall 4, alar facial groove or junction 5, supratip 6, tip-defining points 7, and the philtrum 8.

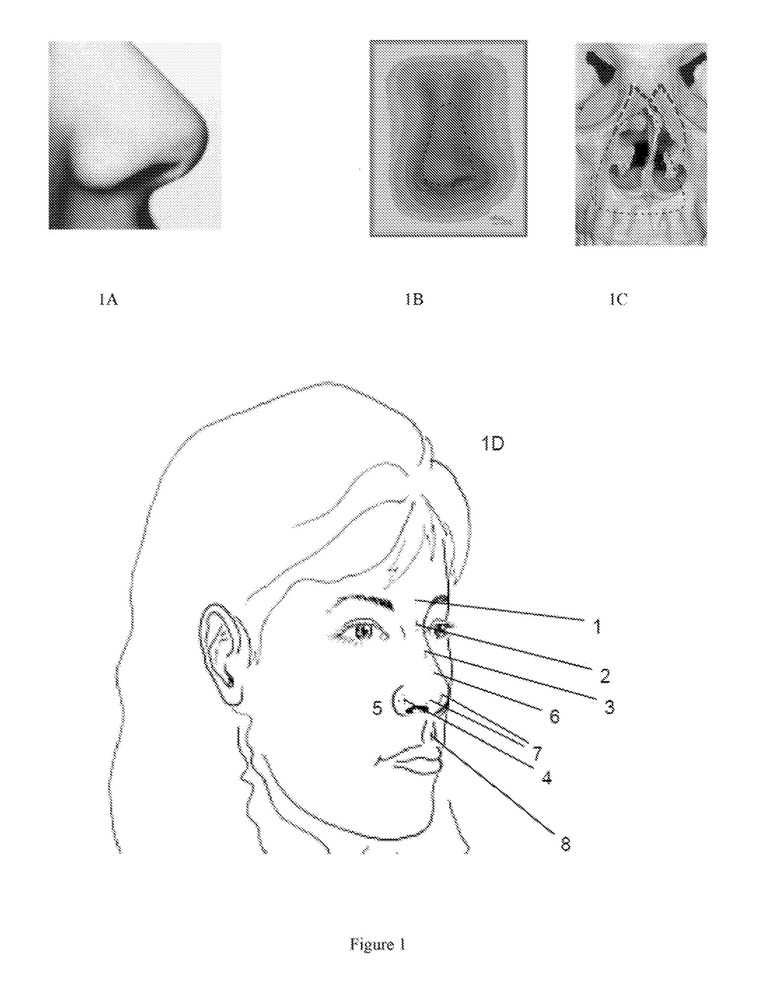

FIGS. 2A, 2B, and 2C depicts various views of the nasal anatomy. FIG. 2A depicts the lateral view of the sagittal plane. FIG. 2B depicts the frontal view of the frontal plane. FIG. 2C depicts the inferior view of the axial plane. Reference number 21 identifies the pyriform aperture. Reference number 22 identifies the septum. Reference number 23 identifies the inferior turbinate.

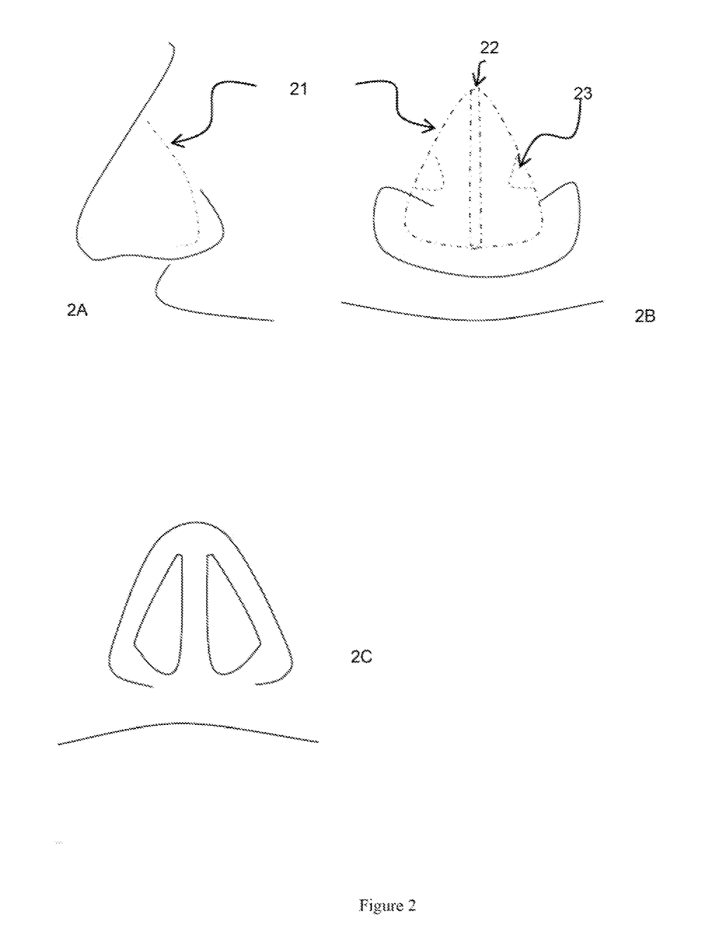

FIG. 3 depicts a lateral view of the nasal bones and cartilage. Specifically identified are the nasal bone 31, the nasion (nasofrontal suture line) 32, internasal suture line 33, nasomaxillary suture line 34, ascending process of maxilla 35, rhinion, or osseocartilaginous junction 36, upper lateral cartilage 37, caudal edge of upper lateral cartilage 38, anterior septal angle 39, lower lateral cartilage lateral crus 40, medial crural footplate 41, intermediate crus 42, sesamoid cartilage 43, and the pyriform aperture 44.

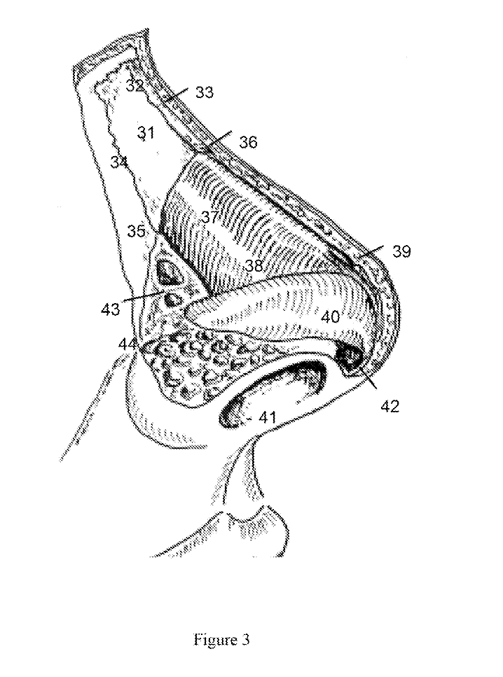

FIG. 4 depicts the septum of the nose. Specifically identified are the quadrangular cartilage 45, nasal spine 46, posterior septal angle 47, middle septal angle 48, anterior septal angle 49, vomer 50, perpendicular plate of ethmoid bone 51, maxillary crest, maxillary component 52, and maxillary crest, palatine component 53.

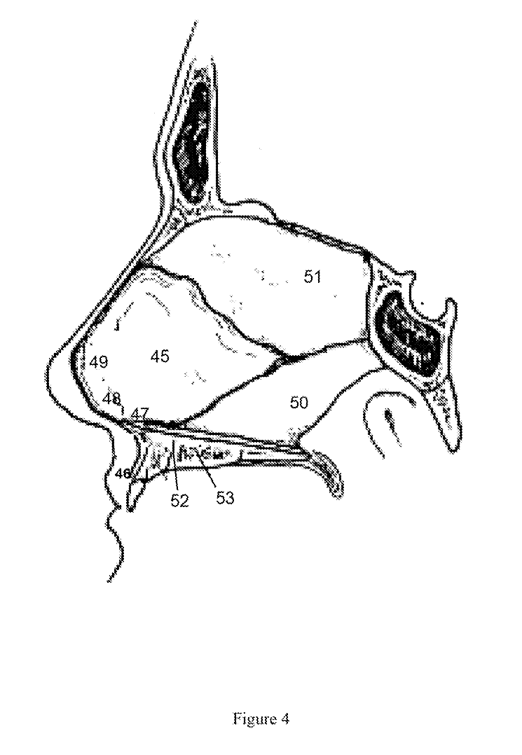

FIG. 5 depicts underlying bones of the intranasal anatomy.

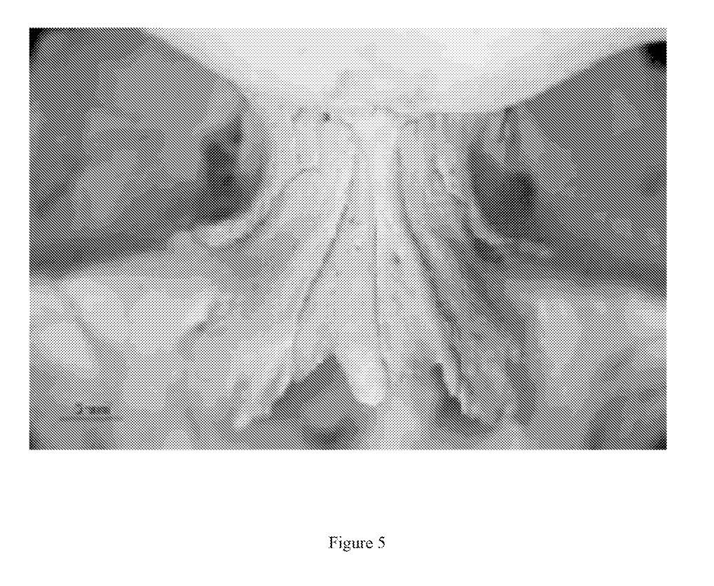

FIG. 6 depicts the lacrimal system. Specifically identified are the punctum 61, canaliculus 62, common canaliculus 63, middle turbinate 64, valve of Hasner 65, inferior turbinate 66, lacrimal sac 67, and the nasolacrimal duct 68. The lacrimal system drains tears from the surface of the eye. Tears enter punctum 61 at the medial aspect of the eyelids and drain into a lacrimal sac 67, then downward to drain into nasal cavity below inferior turbinate 66.

FIG. 7 depicts a coronal section of the nasal cavity. Specifically identified are the superior concha 71, sinus drainage cavities 72, middle concha 73, inferior concha 74, the anterior cranial fossa 75, the orbit 76, the superior meatus 77, middle meatus 78, and maxillary bore 79.

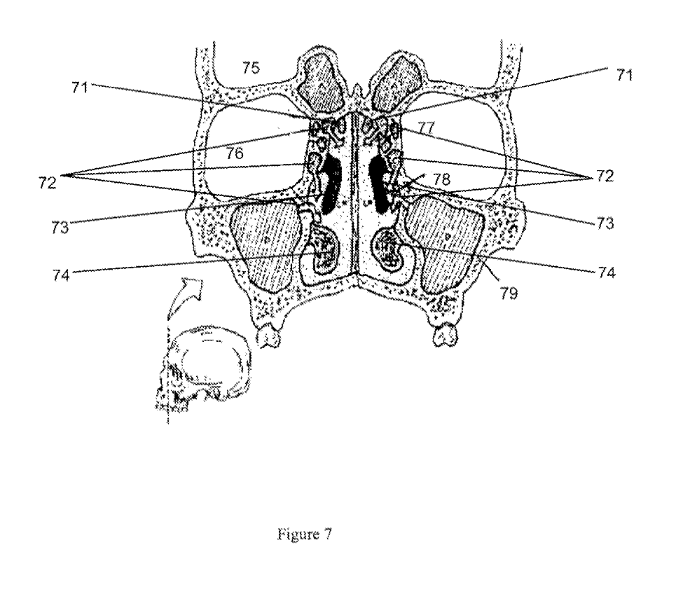

FIG. 8 depicts skin areas of the nose (indicated by cross hatching) where topical diffusible BoNT can be applied. FIG. 8A is a lateral view, FIG. 8B is a frontal view, FIG. 8C is an inferior view, and FIG. 8D is a lateral view. The shaded area of FIG. 8D indicates underlying skin in the intranasal area.

FIG. 9 depicts exemplary needle approaches to the nasal cavity. The darker areas of the needle bore designate that part of the needle that is outside skin. The lighter areas of the needle bore are inside or have passed through tissue. Injection can be made after puncturing skin from outside of the nasal cavity, or the needle tip can be advanced directly to the nasal mucosa. FIG. 9A shows a lateral view of an injection in the nasion that passes through skin and between or through the nasal bones. FIG. 9B is a frontal view of an injection between nasal cartilages. Slight changes in angulation can reach either side of the septum and either lateral nasal wall. FIG. 9C shows an inferior view of a needle entering and passing through nasal alae to reach inferior turbinate. Needle position 91 shows an injection in the nasion that passes through skin and between or through nasal bones. Needle position 92 shows an injection between nasal cartilages. Slight changes in angulation can reach either side of the septum and either lateral nasal wall. Needle position 93 shows the needle entering and passing through nasal alae to reach the inferior turbinate. Needle position 94 shows the needle entering mucosa under the lip and passing across nasal airspace to enter the inferior turbinate. Need position 95 shows the needle entering the skin of the cheek and passing the pyriform edge to reach the inferior turbinate.

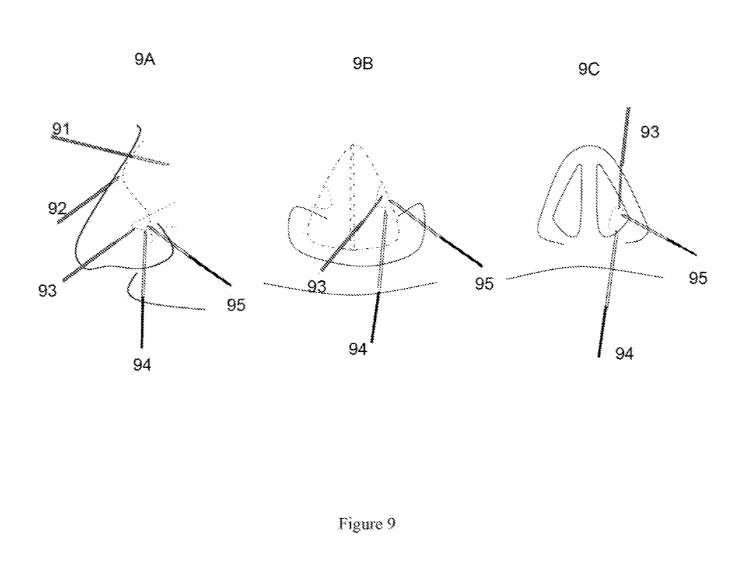

FIG. 10 depicts exemplary transdermal nasal pressure injection. FIG. 10A depicts a lateral view of transdermal pressure injection to the inferior turbinate. FIG. 10B shows a frontal view of transdermal pressure injection to the inferior turbinate. FIG. 10C shows an inferior view of transdermal pressure injection to the inferior turbinate. FIG. 10D shows a lateral view of injection to the nasal septum. FIG. 10E shows a frontal view of injection to the nasal septum. FIG. 10F shows an inferior view of an injection to the nasal septum.

FIG. 11 depicts exemplary deliveries by intranasal spray. Intranasal spray can coat interior mucosa with droplets or particles containing BoNT. FIG. 11A shows a lateral view of delivery by intranasal spray. FIG. 11B shows a frontal view of delivery by intranasal spray. FIG. 11C shows an inferior view of delivery by intranasal spray.

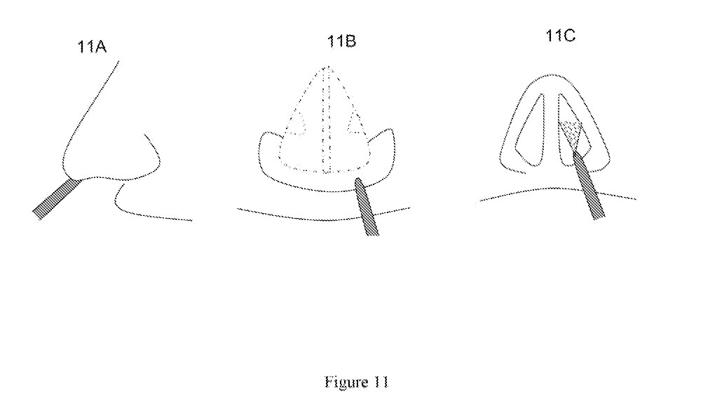

FIG. 12 depicts exemplary delivery by intranasal pressure injection. FIG. 12A shows a lateral view of delivery by intranasal pressure injection. FIG. 12B shows a frontal view of delivery by intranasal pressure injection. FIG. 12C shows an inferior view of delivery by intranasal pressure injection.

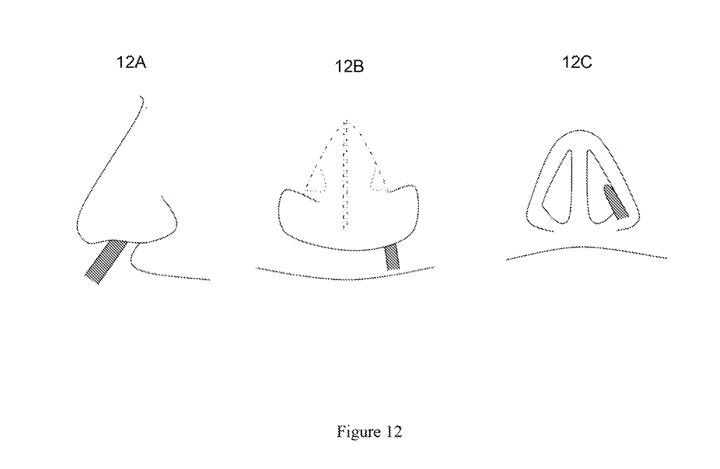

### DETAILED DESCRIPTION

### Definitions

Botulinum toxin (BoNT) refers to botulinum serotypes A, B, C, D, E, F, G. BoNT also encompasses all modified or substituted versions of these toxins that have the same blocking effect on snare proteins. These include any substitution or modification of at least 1 amino acid of a naturally produced toxin or novel toxins made with recombinant techniques. Also included are toxins with removal or substitution of the binding domain and/or translocation domain. Also included are methods of drug delivery including liposomes, protein transduction domains, cationic proteins, acidic solutions and numerous other methods known in the art. These variations are discussed in WO 2004/1076634 and U.S. Pat. No. 7,491,799, which are incorporated by reference in their entirety.

Doses of BoNT described herein are those using botulinum toxin A manufactured by Allergan Inc., Irvine, Calif. (Botox®). Other toxins have known biological equivalence ratios to Botox. Dysport from Ipsen LTD, Bath, England has ⅓^{rd} the bioequivalence per unit of Botox®. Myobloc (Botulinum toxin type B), Solstice Neuroscience, Malvern, Pa. has 1/40^{th} the bioequivalence of Botox®.

Clostridia neurotoxin (CnT) refers to a neurotoxin derived from a species of Clostridia, including, without limitation, C. botulinum (including BoNT as described above), C. butyricum, C. beratti, and C. tetani.

Microneedle injection refers to extremely thin and short solid or hollow needles. Microneedles are solid or hollow needle structures that are much thinner than hypodermic needles. Suitably they are 1 to 100 micron in thickness and They can penetrate the mucosa or skin with little or no pain. The BoNT can be coated on the surface of these needles such that they are physically passed through mucosa and then diffuse off the surface of the needle into tissue. In another embodiment the botulinum toxin can be topically applied to mucosa, and microneedles can then be used to make micropunctures through which the BoNT can diffuse. In another embodiment the microneedles can be hollow and serve as conduits through which BoNT can be delivered (U.S. Pat. No. 6,558,361, incorporated herein by reference in its entirety).

The conduit of a microneedle is very thin so that volumes of 0.1 to 10 ml require many microneedles (from 1 to 1,000,000) and/or delivery over extended periods of time (1 second to 1 week). The hollow microneedles may be attached to a reservoir of drug solution (e.g., U.S. Pat. No. 3,964,482). The flow of drug may be increased by applying pressure (about 0.001 to about 100 pounds per square inch) (e.g., US20090030365) or an electrical field (e.g., U.S. Pat. No. 6,256,533).

Needle injection refers to hypodermic needles. Preferably small gauge needles (e.g. gauge 27-36).

Pressure or Jet injection refers to a method of injection in which pressure injectors propel drug via brief pulses of highly pressurized gas. Examples of pressure injectors are J-tip needle-free syringes, National Medical Products Inc, Irvine, Calif., and SyriJet, Mizzy, Cherry Hill, N.J. (0.2 ml).

U.S. Pat. No. 5,049,125 discloses a device for pressure injection and is hereby incorporated in its entirety.

U.S. Pat. No. 5,630,796, hereby incorporated by reference in its entirety, describes a needleless syringe that delivers pharmaceutical particles entrained in supersonic gas flow from Mach 1 to Mach 8. Optimum particle densities for transdermal delivery range between about 0.1 and 25 g/cm3, and optimal velocities range between 100 and 3000 m/sec.

U.S. Pat. No. 7,060,048, hereby incorporated by reference in its entirety, describes a needleless syringe whereby drug is accelerated by a pressure of no more than 10 bar and penetrates from 10 to 500 microns.

Yamada (Anesth Prog 51:56-61 2004) described using pressure syringes to inject small drugs like epinephrine into nasal mucosa.

Topical application means to be applied to a surface on the body, such as skin or mucosa.

Diffusable formulation means BoNT that can diffuse across mucosa or skin and subcutaneous tissues. These may be BoNT suspended in liposomes or coated by transduction proteins. WO 2006/094263 describes such formulations and is hereby incorporated by reference in its entirety.

Allergic rhinitis (hayfever), non-allergic rhinitis, infectious rhinitis, sinusitis, asthma, COPD (bronchitis and emphysema), migraine headache, impaired cerebral blood flow, headache, sleep breathing disorders, and ocular disorders can each be treated by the application of BoNT to the nose or sinus, or to the nerves innervating these structures. Part of this invention is that BoNT can unexpectedly be applied to areas surrounding these structures, for example, from about 1 to about 100 mm away, and still have a therapeutic effect. Doses of BoNT can, for example, range from about 0.1 to about 1000 units, or from about 1 to about 100 units. Without being bound by a particular theory, the success associated with this treatment may arise from decreased allergic reactions, thinning of mucosa, reversing or decreasing pathological fibrosis of mucosa, and reflect changes in the nose, sinuses, and lungs. The thinning of mucosa increases mucus drainage and allows greater airflow. BoNT causes changes in airway reflexes that improve sleep disordered breathing, asthma, and COPD. BoNT also causes changes in vasomotor reflexes and tone that improve cerebral circulation and tone.

BoNT can be delivered transdermally to nasal and sinus mucosa by topical compositions, needle injection, microneedle injection, or by pressure injection. The BoNT can pass across skin, subcutaneous tissue, and if necessary, bone and cartilage.

When injecting BoNT, injection can be made to the hard palate, especially the nerve foramina.

Asthma can be treated by application of BoNT to intranasal or sinus mucosa, or the nerves innervating these structures. In one embodiment, the BoNT is applied to the nasal fontanels.

Decreased cerebral circulation can be treated by intranasal and sinus application of BoNT. The large surface area and vascularity of the nasal and sinuses distributes BoNT to many vascular neurons. Retrograde transport of BoNT to central neurons distributing to other areas of the head causes relaxation of arteries with increased flow. Vascular headaches are decreased by the same relaxation effect. In some embodiments, BoNT is administered to the sphenopalatine ganglion, where many of these vascular neurons concentrate.

### Drug Delivery Across Skin to the Nasal Cavity and Sinuses

There is a need in medicine for improved drugs and drug delivery methods for nasal and sinus diseases. Disclosed herein are new compositions of nasal drugs and methods of delivering these drugs.

Currently, BoNT has been applied by injections into the mucosa of the nasal cavity or by packing the nose with gauze soaked in BoNT solution. Injection into mucosa with needles is undesirable due to pain, bleeding and risk of contamination. Gauze soaked in BoNT can leak out of the nose and the actual amount delivered is unpredictable. In addition, any procedure within the nose, whether needle injection or topical, would require an Ear, Nose and Throat specialist (otolaryngologist) thereby making the procedure more expensive and harder to obtain than if it could be performed by general medical practitioners.

Surprisingly it has been discovered that drugs, with BoNT being one non-limiting example, can be delivered to the nasal mucosa by application outside the nasal cavity. This can be done safely with less pain and side effects than intranasal injection. Moreover, this method allows broader use and greater patient acceptance.

Drugs delivered by this invention can be those treating nasal and sinus disorders (steroids, antihistamines, nasacrom, antibiotics). In addition this invention includes drugs delivered to the nasal and sinuses intended for systemic distribution; ex insulin; and vaccines.

The areas allowing external application are the nasal skin overlying the nasal cartilages, nasal bones, maxillary, lacrimal, ethmoid, and frontal bones.

In addition, it has been discovered that the lacrimal system can be used to delivery drugs to the nasal cavity as well as to the eyelids and conjunctiva.

In addition it has been discovered that drugs can also be delivered transmucosally to the nasal and sinus cavities. Injection can be performed across the gingival mucosa under the upper lip. The upper margin of the gingival is almost level with the floor of the nasal cavity. More laterally, injections can be made across the gingival mucosa and the anterior wall of the maxillary sinus, particularly in the area of the infraorbital foramen.

In addition, injection can be made into the oral mucosa of the hard palate, where the extensions of the nasopalatine nerves terminate. Injections also can be made where the nerves cross, specifically the foramina.

### Topical Application

The external nasal skin overlaps or is close to intranasal mucosa. Therefore, medication that can diffuse through soft tissue can be applied topically and reach the mucosa. Substances that are capable of this include liposomes and protein transduction domains, and cationic proteins, and nanoemulsions (See, e.g., US20090163412, US20070077259, US20100150994, and US20100172943, each of which is incorporated by reference in their entireties). Preferably, the topical medication is placed on skin overlying mucosa, such as the nasal alae. In this case the diffusion need only travel the thickness of the skin, about 1-2 mm. However, diffusion can travel laterally also, such that it can be placed on the nasal tip and skin around the nose. Therefore the distance of diffusion can vary from about 1 mm to about 2 cm.

### Drug Delivery Across Mucosa

BoNT can also be delivered intra-nasally (including within sinuses) by spraying liquid, gel-like material or solids that adhere to mucosa. These particles, whether drops, gels or solids, may be from about 1 micron to about 1 mm in diameter if aerosolized and inhaled, as they naturally deposit onto the nasal cavity walls. In addition, these liquid, gel or solid particles can be propelled against the nasal wall using a pressure source. The pressure injection can be at a distance of about 0.1 mm to about 200 mm such that the particles pass through air before impacting the mucosa. When propelled by pressure, the lower range of particle size can be about 1 picometer to about 1 micrometer in diameter. BoNT can be contained within particles and can dissolve after application. This allows a simple manner of delivering the drug to nasal mucosa or sinus mucosa.

BoNT can be delivered across mucosa with jet injection, microneedles or by disrupting the mucosal barrier by concomitant application of allergens, chemicals, or energy.

Jet injection uses pressure to propel liquid or particles at great speed. The nozzle of the injector is usually held directly against mucosa and the pressure physically propels the particles across the mucosa. The depth of penetration depends on the design of the nozzle and these parameters are known in the art.

The passage of BoNT over the nasal mucosa can be facilitated by application of allergens, chemicals, osmotic pressure, or energy to open mucosal barriers. In allergic individuals, allergens cause openings between nasal mucosa cells and allow proteins to diffuse across the mucosa. Any substance causing inflammation also opens the mucosal barrier. Certain chemicals are known to cause the same effect. Suitable agents include cationic polymers, bioadhesive agents, surface active agents, fatty acids, chelating agents, mucolytic agents, cyclodextrin, microsphere preparations or combinations thereof (U.S. Pat. No. 5,629,011, U.S. Ser. No. 10/596,817, and U.S. Pat. No. 7,696,343, each of which is hereby incorporated by reference in its entirety). Osmotic pressure aids in transferring drugs across mucosa (U.S. Pat. No. 6,767,901, US20050019314, each of which is hereby incorporated by reference in its entirety). Energy delivered as electrical pulses, electroporation (e.g. U.S. Pat. No. 6,692,456, incorporated herein by reference in its entirety), ultrasound or heat can also be used.

In some embodiments it is preferable that the BoNT carrier be bioadhesive so that it sticks to the mucosa rather than leak away. Bioadhesive compositions are known in the art (U.S. Pat. No. 7,846,478, incorporated herein by reference in its entirety).

### EXAMPLES

### Example 1

### Topical Nasal Application

A 30 year old female has allergic rhinitis as reflected by post nasal drip, congestion and sneezing. She also has allergic asthma reflected by wheezing and coughing.

200 units of diffusible BoNT in 2 cc of a gel carrier are placed on the external nares for 30 minutes and then wiped away. The BoNT diffuses across skin to reach the internal nasal mucosa. After 1 week the patient notices decreased sneezing and nasal itching. Pulmonary function tests show decreased bronchial reaction and coughing.

Alternatively, about 1 cc of a gel carrier can be placed on the skin of the nasal vestibule.

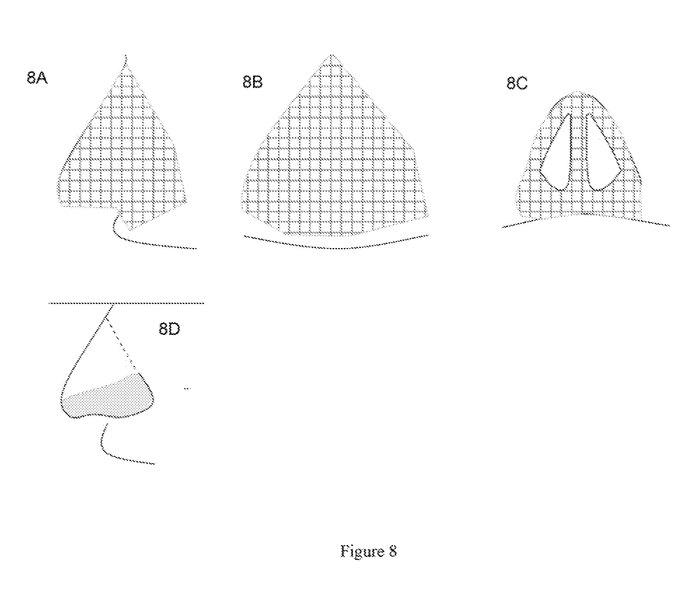

The dermal area that BoNT can be applied is shown in FIG. 8. The preferable mucosa for allergic rhinitis is the most anterior part of the nasal cavity.

### Example 2

### Needle Injection

Needle injection can be performed across external nasal skin in any of the cross hatched areas shown in FIG. 8. Once across skin BoNT can be injected into the soft tissue and allowed to diffuse to the nasal mucosa. However, there is less need for diffusion and more efficient delivery if the needle is advanced to a mucosal target. In some embodiments, the needle can be advanced directly to the nasal mucosa. In other embodiments, the needle may then pass through the nasal mucosa and thereby enter the nasal cavity where it can be redirected to inject multiple areas.

A 20 year old male complains of seasonal sneezing and congestion and is diagnosed with allergic rhinitis. His physician injects 20 units of BoNT mixed with 0.5 cc normal saline into the anterior tip of each inferior turbinate. Specifically the physician feels the patient's face to find the bony margin of the nasal aperture. Then a 1 cc syringe with a 1″ 32 gauge needle is filled with 40 units of BoNT. The needle is inserted at or within the palpated bony margin of the pyriform aperture. The level of the inferior turbinate is just superior to the attachment of the nasal alae to the face. The needle is inserted 4 mm deep and BoNT/saline mixture is injected over 1 min. The needle is withdrawn and the same injection is performed on the opposite side. The patient is then sent home. Follow-up appointment in 2 weeks shows that the patient's sneezing and congestion has greatly improved.

### Example 3

### Transdermal Pressure Injection

Pressure injectors propel drug via brief pulses of highly pressurized gas. Unexpectedly a large molecule such as BoNT can be propelled across skin, subcutaneous tissue, bone, fat and mucosa. Injection can be straight into tissue or at an angle. Injection can remain within tissue or pass into the nasal cavity. The injection depth can vary from 0.1 mm to 20 mm. The volume injected can vary from 0.01 ml to 10 ml. Pressure ranges, particle sizes and speeds are taught in the references cited above.

The nozzle of a pressure injector can be applied at the external nares with the angle facing toward intranasal mucosa; at the pyriform aperture with the angle facing toward intranasal mucosa; over the nasal bones with injection passing through the bones; above the lacrimal sac or duct with injection passing across skin and entering these structures; Under the lip against gingival with the angle toward intra nasal mucosa; under the lip against mucosa with the angle facing directly through the anterior wall of the maxillary sinus (or directed through the foramen); and against the hard palate at the sphenopalatine foramen with the injection going toward the sphenopalatine ganglion.

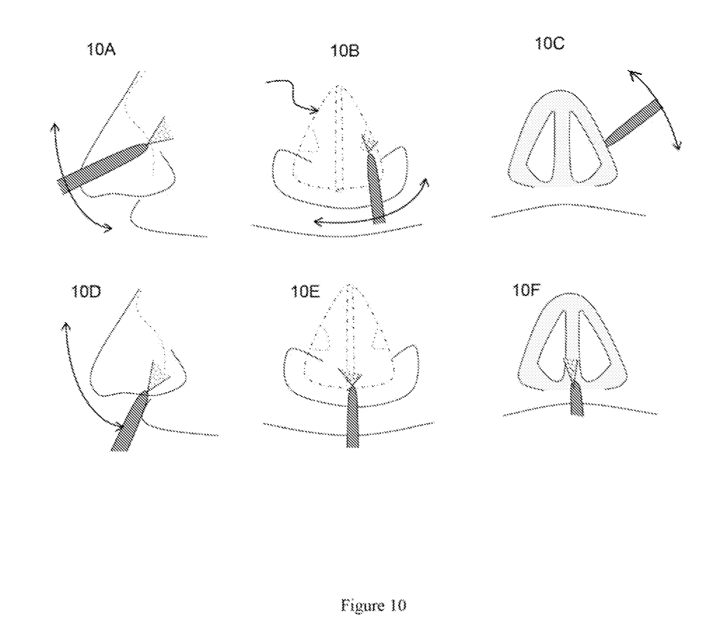

A 50 year old male has chronic rhinitis. A pressure injector nozzle is placed extranasallly on the nasal alae, the shaft is pointed transverse toward the pyriform bony margin. A 20 bar pressure pulse projects a 0.1 ml solution of 10 units of BoNT into the skin and through subcutaneous tissue to end in nasal mucosa in and around the inferior turbinate. As FIG. 10 shows, the nozzle angle can vary.

### Example 4

### Intranasal Spray

The use of aerosol sprays is impractical with botulinum toxin as aerosolized liquid particles can be inhaled into the lungs. However, pressurized droplets or particles that are propelled directly from nozzle to target mucosa circumvent the problem of aerosolized medication. Instead of aerosol the term spray better characterizes this invention. Much like a spray paint, droplets of liquid or gel, or dry particles are sprayed into the nostril. This method is a variant of topical application.

The sprays consist of BoNT within a carrier such as a bioadhesive gel or particles (including nanoparticles). Spray-congealing, consisting of the atomization of a dispersion of the drug in a molten carrier, is a solvent-free technique, which may be advantageous for the preparation of mucoadhesive micro particles. Chitosan, sodium carboxymethylcellulose and poloxamers are examples of carrier materials. Zhou and Donovan used putative bioadhesive polymers such as methylcellulose, sodium carboxymethyl cellulose, hydroxypropyl methyl cellulose, carbopol 934P, chitosan glutamate and pluronic F127 for the study of mucociliary clearance with the use of rat model. The mucociliary clearance for these polymer gels from nasal cavity was determined by following removal of microspheres which are fluorescently labeled and incorporated in the formulation. It was found that the pluronic F127 and other polymer gel formulations have longer residence times and hence decrease mucociliary clearance.

These particles can dissolve in place and release the BoNT. Similarly spray consisting of bioadhesive gels containing BoNT can land on mucosa and release BoNT. These would deliver 0.1 to 100 units, preferably 1 to 20 units as total dose.

A 10 year old male has allergic rhinitis with nasal congestion sneezing and a runny nose. Patient has frequent episodes of sinusitis requiring antibiotics. His physician sprays a poloxamer gel impregnated with 10 units of BoNT into each nostril. The viscous gel is dispersed into particles and land on the surface of the medial and lateral walls. The BoNT dissolves from the particles and reaches mucosa. At a follow-up appointment 1 month later the patient reports diminished sneezing and congestion. There are no episodes of sinusitis for 5 months.

### Example 5

### Intranasal Pressure Injection

Another embodiment of this invention is the use of pressure injection within the nose. This is superior to needle injection as there is no mucosal puncture. The difference between this and nasal spray is that pressure injection is held against tissue and uses higher pressures to force medication into tissue. In some cases the pressure is used to force medication through constricted spaces such as the ostia of sinuses. In still other embodiments the pressure injector is used to inject across thin bone.

A 40 year male complains of perennial rhinitis with significant congestion and post nasal drip. The patient has used topical steroids without relief. Examination shows swollen mucosa bilaterally and mucous rhinorrhea. The physician uses a pressure injector with a 6 cm nozzle. The injector is set to inject a 0.5 cc bolus containing 50 units of BoNT in normal saline. The physician decongests both nasal cavities with 1% neosynephrine spray. Then the nasal mucosa is sprayed with 1% lidocaine. Under direct vision the nozzle is advanced to the posterior superior nasal cavity and pressed against mucosa. The pulse drives the BoNT solution through the thin bone into the pterygopalatine space. Within that space the Sphenopalatine ganglion is blocked, thereby causing a nearly complete block of efferent innervation to the nose and sinuses. In two weeks the patient reports decreased nasal congestion and post nasal drip.

### Example 6

### Lacrimal System

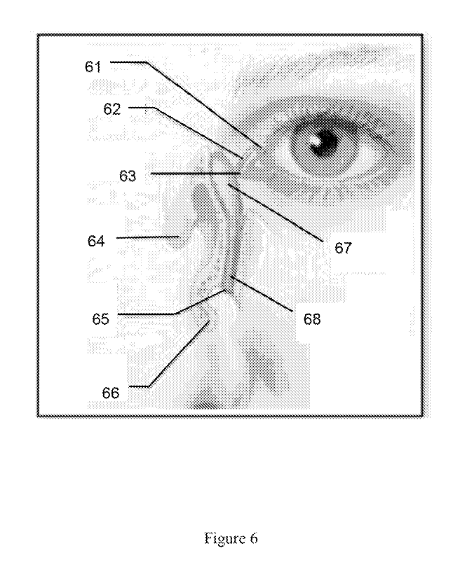

The lacrimal system is illustrated in FIG. 6. The lacrimal system drains tears from the eyes through puncta at the inner aspect of eyelids. These in turn connect to the lacrimal sac, and then drain through a duct to be released into the lateral wall of the nose. The lacrimal system is a convenient conduit for delivering BoNT the nasal cavity. The lacrimal sac is a convenient spot for drug depot, large volumes or drug reservoirs that elute drug over long periods.

BoNT can be delivered to the lacrimal system in a variety of ways. It can be dissolved in eye drops that drain into the punctum. It can be injected or pressure injected through the punctum. It can be delivered by special punctum needles as shown in middle photo. It can be injected or diffuse across skin into lacrimal sac or nasolacrimal duct.

A 40 year old male with allergic rhinitis and allergic conjunctivitis has 10 units BoNT in 0.1 cc normal saline pressure injected across skin into each lacrimal sac. Within 1 week his symptoms have diminished by 50% and this lasts for 4 months.

### Example 7

### External Delivery to Sinuses

Surprisingly, drugs having an effect on the nasal mucosa can be delivered first to the paranasal sinuses. Paranasal sinuses are themselves therapeutic targets, however, as they drain into the nasal cavity they also delivery drugs to the nasal mucosa. Moreover as they drain through the ostia it is of special importance when ostial narrowing or stenosis is an issue. Lastly, sinuses have large surface areas which can be of benefit in certain conditions.

In one embodiment, needle injection can be performed through nasal, ethmoid, frontal, maxillary or palatine bones.

In another embodiment pressure injection can be performed across the anterior walls of the maxillary sinus, particularly at the site of the infraorbital formen. The injection can be done across including the skin of the cheek or under the lip and across the intraoral mucosa.

A 40 year old male with rhinitis and left maxillary chronic sinusitis. A pressure injection of 1 cc of BoNT solution containing 50 units of BoNT is made across the anterior wall of the maxillary sinus in the region of the infraorbital foramen.

### Example 8

### Asthma

Another part of this invention is the treatment of asthma and other lung diseases by application of BoNT to the head and neck, preferably the upper airway and most preferably the nasal mucosa. The amount of BoNT applied can be 0.1 to 1000 units, more preferably 1 to 100 units. BoNT can be applied unilaterally or bilaterally, topically or by needle or pressure injection. The methods described in this application and referenced applications can be used to deliver the toxin to nasal mucosa.

A 20 year old male has a ten year history of asthma. He has end expiratory wheezing most days and has had to come to emergency rooms 3 times in the past year. He currently is taking inhaled steroids. Fifty units of BoNT in 1 cc of normal saline solution is placed on gelfoam pads that are placed in each nasal cavity. Alternatively, twenty units of BoNT could be injected into each inferior turbinate, or twenty units of BoNT could be pressure injected across the nasal wall into the Sphenopalatine ganglion. After 1 week the patient's wheezing diminishes. After 1 month he stops his inhaled steroids and notes no increased difficulty breathing.

A 10 year old male has allergic asthma. Symptoms include wheezing and coughing. Patient has severe exacerbations every month requiring emergency room visits. His physician sprays a poloxamer gel impregnated with 10 units of BoNT into each nostril. The viscous gel is dispersed into particles and land on the surface of the medial and lateral walls. The BoNT dissolves from the particles and reaches mucosa. At a follow-up appointment 1 month later the patient reports diminished wheezing and coughing. There have been no exacerbations this month. Improvement persists for 5 months.

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those show and described herein will become apparent to those skilled in the art and are intended to fall within the scope of the appended claims.

What is claimed:

1. A method of treating a nasal or sinus condition, or a symptom thereof, comprising administering a therapeutically effective amount of botulinum toxin (BoNT) to a selected site outside the nasal cavity of a mammal suffering from a nasal or sinus condition, or a symptom thereof, wherein the nasal or sinus condition, or symptom thereof, is treated.

2. The method of claim 1, wherein the BoNT is administered by needle injection, microneedle injection, jet or pressure injection, or topical application.

3. The method of claim 1, wherein the selected site is selected from the group consisting of: the nasal skin overlying the nasal cartilates, nasal bones, maxillary bones, lacrimal bones, ethmoid bone, or frontal bones; the nasal tip; the external nares; the nasal alae; the palpated bony margin of the pyriform aperture; the lacrimal sac or duct; the eyelid; under the lip; the gingival mucosa; the sphenopalatine foramen; and the infraorbital foramen.

4. The method of claim 1, wherein the BoNT is delivered by needle injection through nasal, ethmoid, frontal, maxillary, or palatine bones.

5. The method of claim 1, wherein the BoNT is delivered by jet or pressure injection across the anterior walls of the maxillary sinus.

6. The method of claim 1, wherein the nasal or sinus condition is selected from the group consisting of rhinitis, infectious rhinitis, allergic rhinitis, sinusitis, asthma, COPD, migraine headache, impaired cerebral blood flow, sleep breathing disorders, and ocular disorders.

7. The method of claim 1, wherein the symptom is selected from the group consisting of nasal congestion, sneezing, rhinorrhea, postnasal drip, nasal or sinus pain, headache, coughing, wheezing, itching, redness, thickened nasal mucosa, and nasal polyp.

8. The method of claim 1, wherein the dose of BoNT is from about 0.1 to about 100,000 units.

9. The method of claim 1, wherein the dose of BoNT is from about 1 to about 100 units.

10. The method of claim 1, wherein the BoNT is combined with a skin permeability enhancing agent.

11. The method of claim 1, wherein the BoNT is combined with a gel.

12. The method of claim 11, wherein the viscosity of the gel increases after administration.

13. The method of claim 11, wherein the gel is a temperature sensitive poloxamer.

14. The method of claim 1, wherein the BoNT is applied with a penetration enhancing agent.

15. The method of claim 14, wherein the penetration enhancing agent is selected from the group consisting of allergen, histamine, and electrical stimulation.

16. A method of treating a nasal or sinus condition, or a symptom thereof, comprising administering a therapeutically effective amount of botulinum toxin (BoNT) by jet or pressure injection to a selected site of a mammal suffering from a nasal or sinus condition, or symptom thereof, wherein the nasal or sinus condition, or symptom thereof, is treated.
